(19) United States
(12) Patent Application Publication
(10) Pub. No.: US 2007/0082018 A1
(43) Pub. Date: Apr. 12, 2007

(54) Stabilized antimicrobial compositions and related methods of preparation

(76) Inventors: Jochen Weiss, South Hadley, MA (US); David Julian McClements, Northampton, MA (US); Eric Andrew Decker, Sunderland, MA (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 11/528,820

(22) Filed: Sep. 28, 2006

Related U.S. Application Data
(60) Provisional application No. 60/721,288, filed on Sep. 28, 2005.

(51) Int. Cl. A61K 47/00; A61K 9/00

(52) U.S. Cl. 424/400; 514/772

(57) ABSTRACT

Antimicrobial compositions and related methods as can be used to enhance stability and/or maintain antimicrobial activity.

This invention claims priority benefit from application Ser. No. 60/721,288 filed Sep. 28, 2005, the entirety of which is incorporated herein by reference.

The United States government has certain rights to this invention pursuant to award no. 2004-35201-15358 from the Department of Agriculture to the University of Massachusetts.

### BACKGROUND OF THE INVENTION

Antimicrobials and antibiotics typically function to inhibit spoilage microorganisms or pathogenic microorganisms (bacterial, yeast and molds), to guard against infection and prevent degradation of a large variety of systems such as foods, pharmaceuticals, cosmetics and biomedical devices. Antimicrobials can range widely in their molecular characteristics and functionality. Generally, compounds can be lipophilic, hydrophilic or amphiphilic (i.e., both hydrophilic and lipophilic). Some compounds act as membrane disruptors by insertion into the bacterial membrane thereby causing leakage (e.g. lysozyme, nisin, natamycin, phenolics such as eugenol and carvacrol). Most of these compounds are amphiphilic and may or may not be charged. Other compounds will diffuse into the cell and disrupt ATP generation by modulating the internal pH of the cell (e.g. organic acids such as lactic acid and acetic acid, and organic acid salts such as sodium lactate and sodium diacetate) or alternatively directly disable the reproductive mechanism of the cell through interaction with the genetic material of microorganisms.

Two arbitrary classifications of antimicrobials are generally recognized: “regulatory approved” (may be synthetic) and naturally occurring. The former includes organic acids (acetic, lactic, propionic), benzoic acid, sorbic acid, nitrites, sulfites, alkyl esters of p-hydroxybenzoic acids (parabens) and some natural antimicrobials including lysozyme, nisin, natamycin and lactoferrin. The latter includes compounds from microbial, plant and animal sources. For example, compounds extracted from the Amaryllidaceae family (e.g. garlic, onion) and the Cruciferae family (mustard, horseradish) have been shown to be particularly effective. Benzylpenicillins and phenoxymethylpenicillins (penicillins G and V, respectively) on the other hand are produced by fermentation and are the basic precursors of a wide range of semi-synthetic antibiotics, e.g. ampicillin. Some compounds, recently found effective, are of particular interest to food systems; for instance, consider chitosan produced by partial or complete deacetylation of chitin extracted from crustacean shells.

These compounds can be applied to materials in a variety of different forms depending on their molecular, functional and physical characteristics (e.g., polarity; that is, polar, non-polar or amphiphilic), solubility (e.g., oil soluble, water soluble or alcohol soluble), molecular organization (e.g., individual molecules or molecular complexes), physical state (e.g., solid or liquid), and physical form (e.g., solid or liquid). In practice, antimicrobials and antibiotics are often incorporated throughout food, cosmetic and pharmaceutical materials by mixing of the pure substances or the substances dispersed in a carrier matrix. Alternatively, these compounds can be applied to the surface to prevent surface growth and contamination from external sources, e.g., using spray coating or dipping procedures.

As identified in the literature, one of the major reasons for the failure to implement more widespread use of antimicrobials and antibiotics is that their efficacy is often low and depends strongly on environmental conditions such as temperature, pH and presence of ionic compounds such as salts. In particular, charged antimicrobials whose functionality depends on electrostatic interactions may loose their activity and stability if the pH and/or ionic strength reach a critical value. For example, organic acids require a low pH to be antimicrobially active. At a low pH the compounds are uncharged and able to diffuse through a bacterial membrane. At a higher pH, after loss of a proton, they become negatively charged. Because the bacterial membrane is also negatively charged, the compound is electrostatically repelled by the membrane, unable to diffuse inside the microbial cell, with resulting loss of activity. Additional problems involve changes in solubility and general dispersion stability, i.e. the compounds can form macromolecular structures or simply fall out of solution.

FIG. 1 illustrates the prior art in more detail, and general problems that may be encountered with amphiphilic antimicrobials if environmental conditions are altered. FIG. 1A illustrates the case where antimicrobials exist as simple dispersions in the system to which they are applied. Upon a change in pH, addition of ionic or oppositely charged compounds and a change in temperature, the compounds can loose their solubility or form macromolecular aggregates that fall out of solution. In this case, the system will separate into two phases. FIG. 1B illustrates the case where the antimicrobial exists as a colloidal dispersion (if concentrations are above the critical micelle concentration). Upon change in pH, addition of ionic or counter-ionic compounds and change in temperature, the colloidal particles will aggregate and form large macromolecular structures and eventually phase separate.

In both cases, destabilization can often be visually observed. The solutions or dispersions are initially transparent and thermodynamically stable. Typically, the system becomes first turbid, then forms a turbid lower layer rich in aggregated antimicrobials and a (clear) supernatant layer that is void of antimicrobials (FIG. 1C). Either case poses significant application problems as it leads to a change in the physicochemical properties of the bulk system that is perceived as a quality defect. More importantly, this is accompanied by a loss in antimicrobial functionality, a serious condition as growth of microorganisms becomes possible.

### SUMMARY OF THE INVENTION

In light of the foregoing, it is an object of the present invention to provide antimicrobial compositions and/or methods for their preparation and/or assembly, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It is an object of the present invention to provide one or more stable antimicrobial compositions, such stability as can be expressed in terms of reduced overall net charge, as compared to one or more components used in the preparation of such compositions.

It can be a related object of the present invention to provide such a composition which, by comparison with a precursor antimicrobial compound, is relatively less affected by environmental change and pH, temperature and/or ionic strength.

It is an object of the present invention to provide one or more stable antimicrobial compositions, such stability as can be expressed in terms of reduced chemical change or degradation when provided in conjunction with one or more surface active components.

It can be another object of the present invention to provide such compositions or related systems exhibiting antimicrobial activity or enhanced functional performance over a wide range of application or end-use conditions.

It can be another object of the present invention to provide such compositions or related systems exhibiting enhanced phase stability, as can be expressed in terms of reduced physical separation from a liquid, solid or semi-solid medium.

It can be another object of the present invention to maintain or preserve compositional or product visual and functional quality over a wide range of temperature, ionic strength or pH conditions.

Other objects, features, benefits and advantages of the present invention will be apparent from this summary and the following descriptions of certain embodiments, and will be readily apparent to those skilled in the art having knowledge of various antimicrobial compounds, compositions and related preparation or formulation techniques. Such objects, features, benefits and advantages will be apparent from the above as taken into conjunction with the accompanying examples, data, figures and all reasonable inferences to be drawn therefrom.

In part, the present invention can comprise a composition comprising a first component capable of antibiotic and/or antimicrobial function under application or end-use conditions, such a first component (a) substantially unassociated or non-self-assembled in a particular medium, or (b) self-assembled or associated in a medium at a certain minimum concentration; and a second component capable of surface-active function, such a second component (a) assembled or associated in the medium at a certain minimum concentration with first component (a) or (b), or (b) substantially unassociated or non-self-assembled at a concentration lower than a certain minimum, with first component (a) or (b). Regardless of concentration, contact of such a second component can be used or is at least partially sufficient to maintain the activity of the first component.

With regard to either the first or second components of such a composition, a certain minimum concentration of the respective component can refer to a concentration providing a thermodynamically-stable dispersion or suspension of a self-assembled or associated component in a particular medium. For those components associated or assembled in a micellar configuration, such a minimally-sufficient concentration can be referred to as a critical micelle concentration, for that medium. For various other such first and second components, thermodynamic stability can be understood with respect to the presence of any such component in a medium without substantial phase separation.

Without limitation, the structural and/or chemical characteristics of each such first and second components can define its interaction with a particular medium and one with another. In certain embodiments, each respective first and second components can be amphiphilic, regardless of charge, induced dipole or hydrogen bonding. In certain other embodiments, one of or both first and second components can comprise a net charge under particular medium conditions in conjunction with hydrophobic/lipophilic character. Such first and second component compositions are limited only by way of resulting association, assembly and/or thermodynamic stability in a particular medium, as compared to the less stable of the components.

In certain embodiments, such compositions can comprise an amphiphilic first component, whether substantially associated or self-assembled or substantially unassociated or non-assembled in a particular medium, having a net positive charged portion (e.g., cationic), and a second amphiphilic surface active component, self-assembled or associated in the medium, having a net negative charged portion (e.g., anionic) or as can be substantially uncharged (e.g., non-inonic). Conversely, in certain other embodiments, such compositions can comprise either an associated/self-assembled or a substantially unassociated or non-self-assembled, amphiphilic first component having a net negative charged portion (e.g., anionic) and a second amphiphilic surface active component, self-assembled or associated in the medium, having a net positive charged portion (e.g., cationic) or as can be substantially uncharged (e.g., non-ionic). Without limitation as to component association, assembly or configuration, in such embodiments, the first component and/or resulting composition can have a reduced net charge, as compared to initial respective component charge(s), such that composition stability and/or microbial activity is maintained or at least less susceptible to environmental change in pH, temperature and/or ionic strength.

In certain other embodiments, such compositions can comprise an amphiphilic first component, whether substantially unassociated or non-assembled or self-assembled or associated in a particular medium, having a net positive charged portion (e.g., cationic) and a second amphiphilic surface active component, substantially unassociated or non-assembled in a particular medium, having a net negative charged portion, (e.g., anionic), or as can be substantially uncharged (e.g., non-ionic) under medium conditions. Conversely, in various other embodiments, such compositions can comprise a first amphiphilic component, either unassociated/non-assembled or self-assembled or associated, having a net negative charged portion (e.g., anionic) and a second amphiphilic surface active component, substantially unassociated or non-assembled in the medium, having a net positive charged portion (e.g., cationic) or as can be substantially uncharged (e.g., non-ionic). Likewise, in such embodiments, without limitation as to component association, assembly or configuration, the first component and/or resulting composition can have a reduced net charge, as compared to initial respective component charge(s), such that compositional stability and/or microbial activity is maintained or at least less susceptible to environmental change in pH, temperature and/or ionic strength.

As would be understood in the art, such compositions can be present, or as formed in a fluid or liquid medium or, alternatively, as can be formed or subsequently incorporated into a solid or semi-solid medium or related matrix material. Representative fluid/liquid media can, without limitation, be aqueous, alcoholic or hydrophobic/lipophilic, and in certain embodiments be used as a carrier component. Representative solid media can, without limitation, include food components or products and carrier, binder or related components of the sort typically found in a wide range of medical, pharmaceutical, food, cosmetic and personal care products.

In part, the present invention can also comprise a method of preparing an antimicrobial/antibiotic composition. Such a method can comprise providing a first antibiotic/antimicrobial component (a) or (b) as described more fully above; and contacting a second surface-active component (a) or (b), with either first component. Such contact can be provided in a particular medium, wherein each respective first and second component can interact with one another and/or the medium as described above. A composition resulting therefrom can be introduced to a subsequent medium, carrier or matrix material, isolated for subsequent incorporation, or applied to a substrate component to impart antibiotic/antimicrobial properties thereto.

In part, this invention can also comprise a method of using a surface active component to maintain antimicrobial activity. Such a method can comprise providing an antimicrobial component in a medium; and contacting such a component with a surface active component. The surface component can be in an amount at least partially sufficient to maintain the activity of the antimicrobial component over change in medium pH, temperature and/or ionic strength. Regardless, such contact can be of the sort at least partially sufficient to stabilize the antimicrobial component in such a medium for: example, where the antimicrobial component is at a concentration sufficient for micelle formation in the medium. Where the antimicrobial component is ionic, such contact can be at least partially sufficient to reduce the net charge of the antimicrobial component.

Accordingly, as demonstrated below, the present invention provides a range of stabilized antimicrobial compositions and methods for their use and preparation, such compositions as can be used in a variety of applications ranging from the food, to the pharmaceutical, to the personal care product industry. In particular, as available through certain embodiments of this invention, currently available component compounds (e.g., without limitation, lauric arginate compounds as may be formulated, and various polyoxyethylene sorbitans) can be used without need for further, specific regulatory approval. As this invention addresses certain deficiencies in the art, its use and implementation should result in substantially increased use of antimicrobials and realization of the benefits available therefrom.

### BRIEF DESCRIPTION OF THE DRAWINGS

FIG. 1. Illustrating the prior art, instability of charged antimicrobials and antibiotics upon change in pH and concentration of ionic compounds leading to aggregation and eventual phase separation.

FIG. 2. Formation of mixed antimicrobial systems comprising micelle-forming surfactants and antimicrobials, in accordance with this invention.

FIG. 3. LAE Structural formula Empirical formula C₂₀H₄₁N₄O₃Cl, representative of other ionic antimicrobials.

FIG. 4. Chemical molecular structure of Tween-20, representative of other nonionic surfactants. Molecular formula C₅₈H₁₁₄O₂₆.

FIG. 5. Evolution of IFT versus concentration of Mirenat, representative of but one possible LAE form or formulation, in accordance with this invention. Brackets indicate the standard deviations.

FIG. 6. LAE micelles size depending on the CaCl₂ concentration. Each solution contains 5% Mirenat v/v and different amounts of CaCl₂.

FIG. 7. Micelles size distribution in volume for pure and mixed systems at pH 4.00.

FIG. 8. Graphic illustration of surface tension as a function of total surfactant concentration.

FIG. 9. Titration of pure and mixed systems: pH versus amount of hydroxide ions added to the systems.

FIG. 10. Plots showing the dependence of the absorbance on the pH at 600 nm. Reference is Mirenat 5% v/v. Others are Mirenat 5% v/v mixed with T20.

FIG. 11. Evolution of the size of lauric arginate micelles depending on the pH. Solutions are composed of 5% Mirenat v/v.

FIG. 12. Plots showing the dependence of the percentage of solids held in 0.45 μm filters with Mirenat 5% v/v and different concentrations v/v of Tween20.

FIG. 13. Mixed micelles size depending on the pH. Each solution contains 5% Mirenat and 5% T20 v/v.

FIGS. 14A-F. Electronic images obtained by microscopy observations for different LAE systems under different conditions. Magnification factor is ×200. A. Mirenat 5% v/v pH 4.02; B. Mirenat 5% v/v pH 11.00; C. Mirenat 5% v/v+T20 0.5% pH 5.02; D. Mirenat 5% v/v+T20 0.5% pH 11.01; E. Mirenat 5% v/v+CaCl2 30 g/L; F. Mirenat 5% v/v+NaCl 30 g/L.

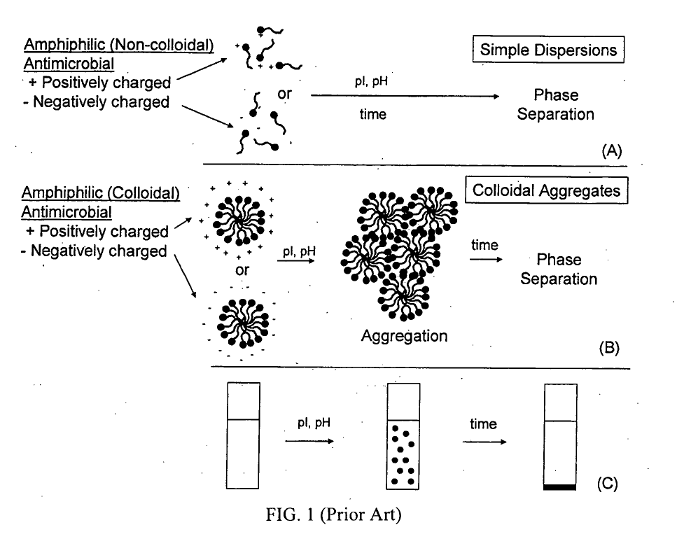

### DETAILED DESCRIPTION OF CERTAIN EMBODIMENTS

Illustrating certain nonlimiting embodiments of this invention, colloidal compositions comprising a surfactant component and an antimicrobial or antibiotic component can be prepared from any combination of the following:

Any antimicrobial capable of forming a mixed micellar system in combination with a surfactant can be used. In particular charged, amphiphilic antimicrobials such as lauric arginate that suffer from stability issues in systems containing ionic compounds can be considered in the context of this invention. Nevertheless, the invention may also be applicable to protein or polypeptide antimicrobials such as nisin, nata-nisin, lysozyme, organic and/or amino acids and their salts and essential oil components, including those phenolic or polyphenolic compounds.

Any surface-active compound capable of forming a colloidal dispersion and that will similarly form a colloidal dispersion with or would otherwise stabilize the antimicrobial would be suitable. This includes the entire range of known and available micelle-forming surfactants. Depending on the nature of the antimicrobial, the surfactant could be anionic, cationic or nonionic. Without limitation, such surface active components for example, acetic acid esters of monogylcerides (ACTEM), lactic acid esters of monogylcerides (LACTEM), citric acid esters of monogylcerides (CITREM), diacetyl acid esters of monogylcerides (DATEM), succinic acid esters of monogylcerides, polyglycerol polyricinoleate, sorbitan esters of fatty acids, propylene glycol esters of fatty acids, sucrose esters of fatty acids, mono and diglycerides, fruit acid esters, stearoyl lactylates, polysorbates, starches, sodium dodecyl sulfate (SDS) and/or combinations thereof. Other such components capable of such surface active function are as provided or inferred in co-pending application entitled “Encapsulated Emulsions and Methods of Preparation”, filed contemporaneously herewith, the entirety of which is incorporated herein by reference. Generally, combinations of nonionic or charged surfactants with charged antimicrobials are expected to be more salt, pH and temperature tolerant than the antimicrobials, alone, since the overall charge of the mixed micelle is reduced.

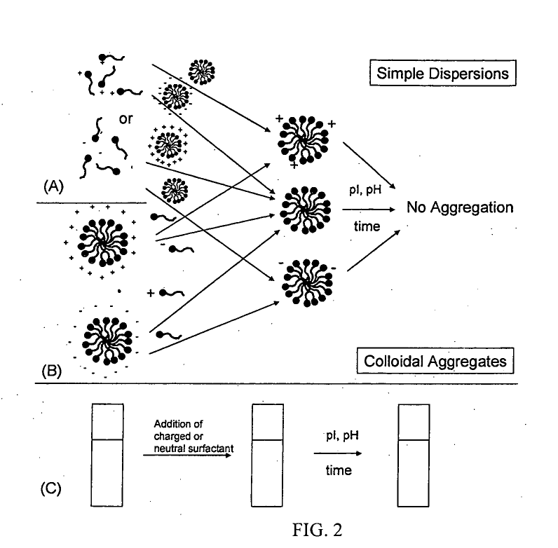

FIG. 2 illustrates several different component combinations useful in the practice of this invention. FIG. 2A assumes that antimicrobials do not form thermodynamically stable colloidal dispersions by themselves. In this case, addition of supramicellar solutions of cationic, nonionic or anionic surfactant micelles will result in the production of mixed micelles that may be neutral, positively or negatively charged. These systems are expected to be significantly more salt, pH and temperature tolerable. FIG. 2B assumes that antimicrobials by themselves form colloidal aggregates (such as the lauric arginates). In this case, addition of submicellar surfactant concentrations will lead to formation of similar mixed micellar structures that again would be substantially more stable and active than their single species counterparts. In terms of optical behavior, these systems can be expected to be transparent at all times (FIG. 2C).

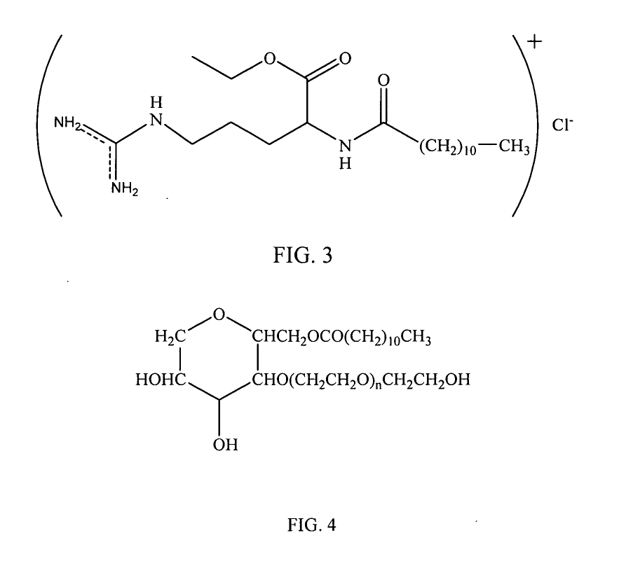

As relates to certain non-limiting embodiments, lauric arginate (LAE), a novel cationic surfactant, is a derivative of lauric acid, L-arginine and ethanol chemically known as ethyl lauroyl arginate HCl (INCI name) or ethyl-N^{alpha}-dodecanoyl-L-arginate hydrochloride (IUPAC name). See FIG. 3. LAE is a broad spectrum antimicrobial with inhibitory activity against Gram-positive and Gram-negative bacteria, yeast and fungi. Minimum inhibitory concentrations against common pathogens e.g. Escherichia coli and Listeria monocytogenes have been reported to be less than 10 ppm, which is an order of magnitude lower compared to the MIC of other food approved antimicrobials such as nisin, methyl and propyl paraben, essential oil compounds (thymol, carvacrol, eugenol), and organic acids and their salts. The high antimicrobial activity of lauric arginate has been attributed to the strong electrostatic binding to negatively charged cell membranes of spoilage organisms and pathogens disrupting the membrane integrity of the cells causing leaking and loss of transmembrane potential which may be followed by cell death.

LAE has attracted increased attention due to its recently approved GRAS status. The compound is readily metabolized to yield the naturally occurring amino acid arginine, which is further broken down to ornithine and urea. Due to the rapid hydrolytic degradation in both liver and plasma, exposure to LAE in vivo is very short and health impacts are thought to be minimal. Mirenat®-N, the commercially available form of lauric arginate, is a formulation of 25% (w/w) of LAE in propylene glycol, a commonly used stabilizer. Levels for application in food systems can range from about 0.5-about 2 g/L.

Like many other ionic or nonionic surfactants, lauric arginate spontaneously self-assembles to form spherical association colloids, so called micelles, when dispersed in a suitable solvent above a critical concentration. Below the critical micelle concentration (CMC), surfactant monomers are present as single molecules dispersed throughout the solvent phase. Contrary to nonionic surfactants, the formation and stability of ionic micelles depends on pH and ionic strength. As discussed more generally, above, addition of salts such as NaCl or CaCl₂, may induce shape transformations of the association colloids from for example spherical particles into larger cylindrically shaped aggregates. These aggregates may be large enough to scatter light thereby inducing turbidity in the solution. In other instances, ionic micelles have been reported to aggregate and subsequently precipitate if counterions are added to the system. Precipitation of surfactant micelles is typically associated with loss of functionality such as detergency, emulsification and foam stabilization and hence is undesirable in formulations where this particular functionality is required. Since many foods may contain a substantial amount of salt and/or are formulated over a wide range of pH values, addition of ionic surfactants such as lauric arginate to foods may lead to destabilization of the colloidal dispersion.

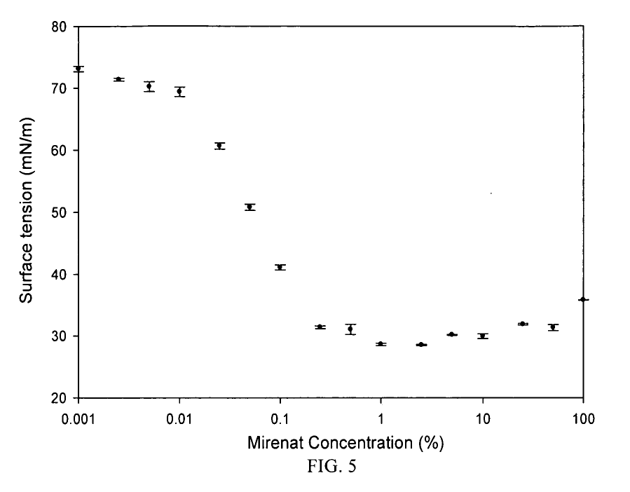

With reference to examples 3-6, below, LAE was studied with and without the presence of a non-ionic surfactant (e.g., Tween 20® (T20); see example 3 and FIGS. 3-4). Surface tension of pure bidistilled water was found to be 71.57±0.57 at 25.9° C. While the concentration of Mirenat is increasing, the IFT tends to decrease, but not linearly (FIG. 5). Indeed, at very low concentrations of Mirenat, there is no change in surface tension: too few monomers are added to cover the whole surface. When additional surfactant is added, surface tension decreases linearly: monomers go to the surface and arrange themselves, decreasing the surface tension. After a certain concentration of Mirenat, there is no further change in surface tension: the surface is completely loaded with surfactant.

The critical micelle concentration of the pure systems was determined by plotting surface tension values against the logarithm of the total surfactant concentration. In order to determine the values of the critical micelle concentration, two linear fits were used. The first line was fitted to interval of concentration characterized by linear decrease of the surface tension and the second one to the region of concentration with the nearly constant surface tension. The point in which the fitted lines cross corresponds to the value of the critical micelle concentration (CMC). Here, the CMC is reached at a Mirenat concentration 0.25% v/v. This corresponds to 1.65 mmol.L⁻¹ of lauric arginate, 4 times lower than the literature value. Accordingly, all experiments will be carried out at a concentration largely exceeding the found value: 5% v/v.

Several experiments were conducted to examine the influence of salts on the stability of solutions containing Mirenat. The pH was not controlled here, as the buffer could mask the real behavior of the system. Still, it remained almost unchanged after the addition of salts. The added counterion (anion) contributed by the added electrolyte is assumed to be the same species as that contributed by the cationic surfactant, i.e. chloride ions Cl⁻. Therefore, chloride salts, i.e. sodium chloride NaCl and calcium chloride CaCl₂, were used. This resulted in final solutions containing 5% Mirenat v/v and 0 to 200 g.L⁻¹ NaCl or 0 to 200 g.L⁻¹ CaCl₂, were used. Precipitation occurred slowly, especially with low salt concentration where it could take up to 5 days. This pace resulted in few regular particles which could be several millimeters long. Almost every time precipitation took place, little or even no turbidity was noticeable. The results provided in Table 1 reflect qualitative visual observation.

TABLE 1
Effect of salts on surfactant mixtures. Observations were taken 72
h after addition of salts. C: clear. P: presence of precipitates

| | | Mir 5% + | Mir 5% + | Mir 5% + |
|---|---|---|---|---|
| Salts | Mir 5% | T20 0.5% | T20 1% | T20 5% |
| NaCl 2 g/L | C | C | C | C |
| NaCl 5 g/L | C | C | C | C |
| NaCl 10 g/L | P | C | C | C |
| NaCl 50 g/L | P | P | C | C |
| NaCl 100 g/L | P | P | C | C |
| NaCl 200 g/L | P | P | P | C |
| CaCl₂ 2 g/L | C | C | C | C |
| CaCl₂ 5 g/L | C | C | C | C |
| CaCl₂ 10 g/L | C | C | C | C |
| CaCl₂ 15 g/L | P | C | C | C |
| CaCl₂ 50 g/L | P | C | C | C |
| CaCl₂ 200 g/L | P | P | C | C |

From data for filtration of precipitated products in systems to which varying amounts of sodium chloride or calcium chloride were added, it is evident that precipitation itself was responsive to salt additions. Precipitated product amounts increased significantly when each of the salts was added, indicating that certain dissolved coupling products become more precipitable as background ion concentrations increase. It should be noted that the precipitated mass was very low, representing only few percent fractions of the total solution mass.

Such results can be explained from the fact that Mirenat is a cationic surfactant with a positively charged hydrophilic head; therefore, micellar surface is positively charged, as confirmed by the positive value of the zeta potential of micelles. When ionic particles of same sign are approaching each other, they electrostatically repel each other, thus keeping particles apart, resulting in a stable suspension. This implies that the interparticle electrostatic repulsion is sufficient to prevent any aggregation occurring. In this region the electrostatic force is strong sufficiently to keep the cationic molecules enough far away to each other. The destabilizing effect of salts can be explained by the charge screening effect of Cl⁻ anions. Under such conditions, counterions are electrostatically bound to the micelles, creating a loss of global electrostatic positive charge for each micelle.

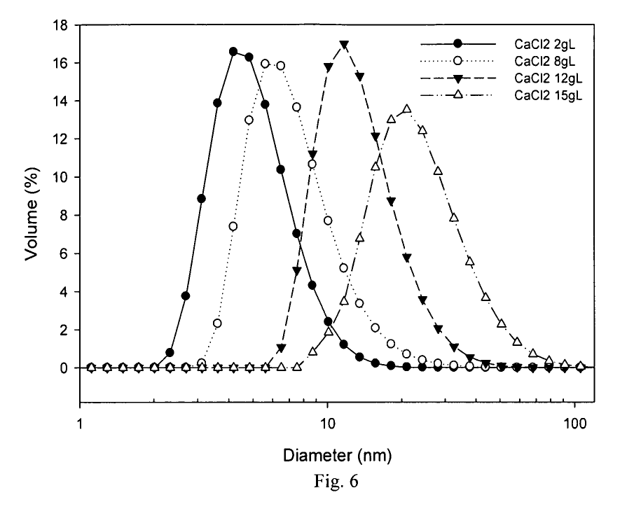

When the counterion amount goes beyond the saturation limit of the micelles, precipitation occurs. This loss of electro-repulsion between structures is seen as a charge neutralization precipitation. Indeed, particles come closer to each other. The electrostatic repulsion force that tends to separate them is not sufficient to offset the van der Waals force. As a result, the particles tend to attract toward each other, making the suspension unstable and thus precipitation occurs. Regarding the micelles size (FIG. 6), distribution in presence of electrolytes shifts to the right as salt concentrations are increasing. The same shape is observed in presence of NaCl. The charge neutralization by adding counterions also decreases the effective head group area for the surfactant monomer to occupy in the micelle and hence the increase in the aggregation number of the micelle, leading to a bigger size.

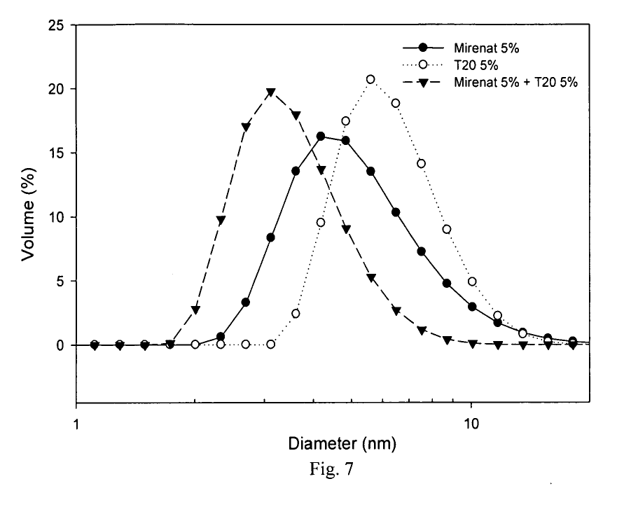

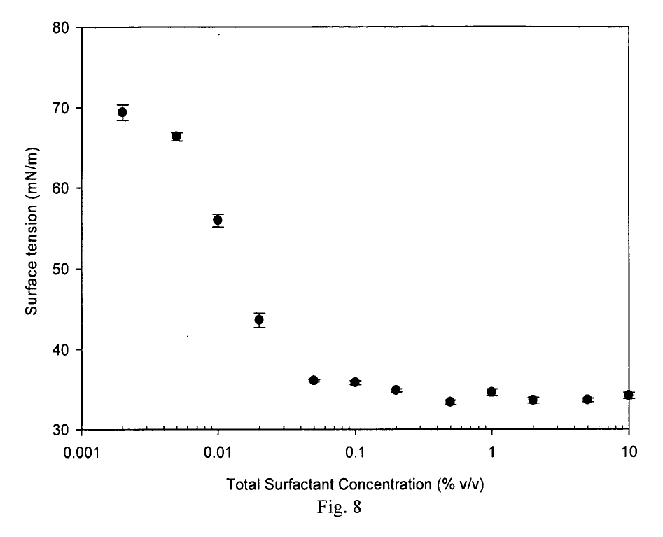

However, precipitation was avoided at high salt concentrations if a nonionic surfactant was added to the medium. Consider the apparent size distributions of pure and mixed systems shown in FIG. 7. Each pure surfactant system exhibits ability to form micelles. Ionic (LAE) micelles (CMC=0.25% v/v) are smaller than nonionic (T20) micelles and their CMC is quite the same as T20 (respectively 0.25% v/v and 0.3% v/v, figure. not shown). This size corresponds to the different length of the hydrophobic tails. When the two components are put together in the same aqueous media, both previous pure micelles disappear and a new category of compound with a smaller hydrodynamic radius appears, and is believed a reflection on the existence of mixed micelles composed of cationic and nonionic particles.

The nonionic shielding can be considered as unexpected. With increased amounts of T20, more and more non-charged surfactant molecules join the ionic micelles. This results in a lower zeta-potential (Table 2). The electrostatic repulsive effect is not as high as with the pure ionic micelles, but counterion binding is believed to decrease upon the addition of nonionic surfactant: binding decreases upon the addition of nonionic surfactant because of the decrease in the surface charge density on the micelle, and micelle screening is lowered.

TABLE 2
Influence of the addition of T20 on the zeta-potential of the micelles.
Concentrations are v/v. When non indicated, pH is 4.00.

| | Mir 5% | Mir 5% + T20 0.5% | Mir 5% + T20 1% | Mir 5% + T20 5% |
|---|---|---|---|---|
| ZP (mV) | 40.38 | 26.42 | 22.71 | 21.11 |

| | Mir 5% pH 2.80 | Mir 5% pH 3.00 | Mir 5% pH 4.00 | Mir 5% pH 3.25 |
|---|---|---|---|---|
| ZP (mV) | 60.97 | 58.05 | 40.38 | 41.163 |

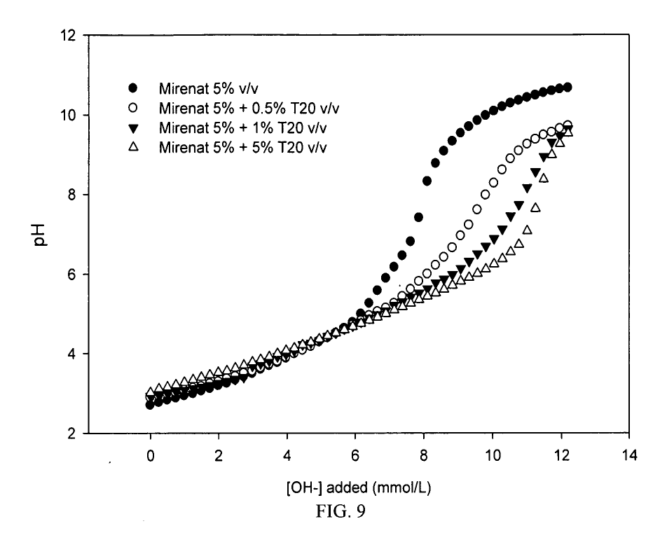

FIG. 9 shows the result of the titration of solutions containing Mirenat 5% v/v and different concentrations v/v of T20. The solution pH was increased stepwise by adding 0.5N NaOH and was recorded 2 min following each addition. It was plotted against the total amount of OH added for each of the solutions. Three minutes following each addition, a sub-sample was delivered via pipette into a cuvette for light absorption determination. Every system exhibits a slight increase of pH in the early steps of sodium hydroxide addition, followed by a pH jump, i.e. a fast and deep break in the pH curve. However, this drastic change doesn't occur at the same hydroxide ions concentration for each of the different T20 concentrations. Indeed, it progressively shifts to the right of the graphic. Notice that beginning pH values are very close to each other for each solution. To the contrary, final pH values corresponding to the addition of 11 mmol OH⁻ are not the same and tend to decrease while the amount of T20 becomes higher. Such results can be related to enhanced buffer capacity as nonionic compound amount is raised.

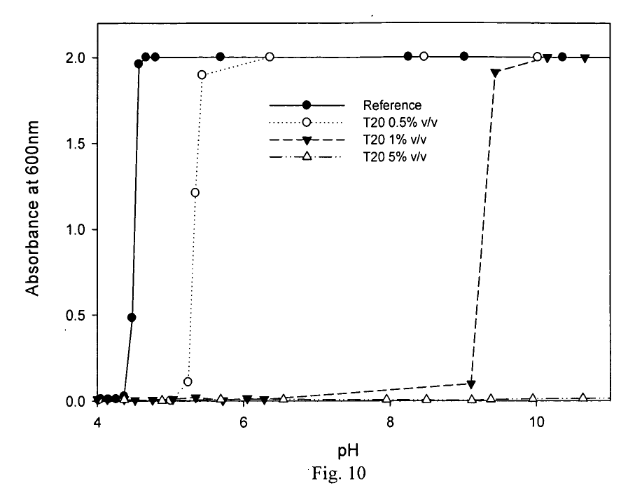

pH would be expected to have an effect on precipitation as it affects the concentration of background ions H⁺ and OH⁻ in the medium. Hydroxide would be expected to interact with the surfactant monomers and micelles, as the chloride counterion did. In FIG. 10, light absorption at 600 nm was plotted against pH for each of the pure and mixed solutions. It has been assumed that the initial rate of change in absorbance in the dispersions is a qualitative measure of the precipitation rate. Light absorption rises with an increase of suspended particle density.

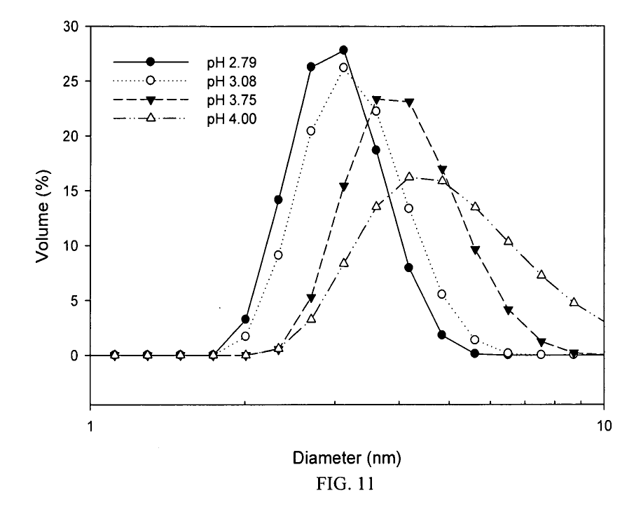

Regarding the pure component curve, the light absorbance deeply increases above a pH value around 4.3, showing a tremendous instability of the dissolved components. Regarding the other systems, a similar drastic change is observed for two of the three mixed systems but above relatively higher pH values than for the pure cationic solution (5.2 and 6.3 for 0.5 and 1% T20, respectively). No significant absorbance modification is remarkable for the mixed system comprising 5% Mirenat and 5% T20 v/v. Note that contrary to the salt experiments, precipitation was immediately visible. Indeed, pH has a direct effect on the soluble ions. With increasing pH, the cationic surfactant, loses its positively charge, and finally loses its solubility. This is indicated by the decrease of the zeta-potential (Table 2). Under such conditions, they become unable to show their repulsive effect. This lack of electrostatic repulsion among neighboring micelles may lead to aggregation, as shown by the micelles size dependency on pH. See, e.g., FIG. 11, where lauric arginate micelles increase in size while the pH of the bulk aqueous medium is increased.

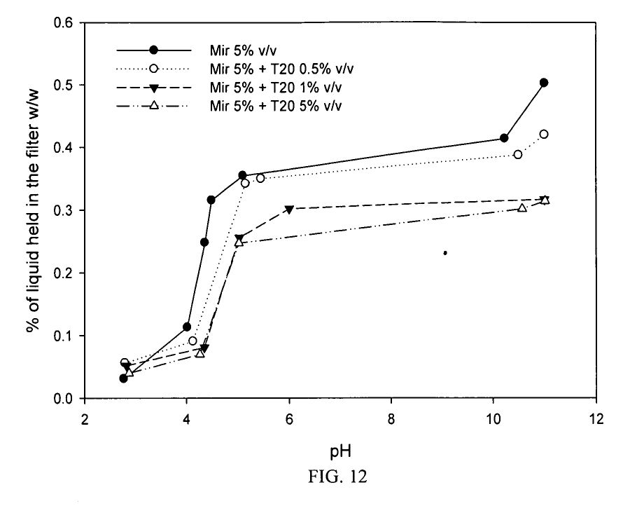

A similar relationship was found between particles held by the filter and pH in the different suspensions, as shown in FIG. 12. It is interesting to notice that even if no precipitation was detected by the turbidimetric study for mixed solutions containing 5% v/v T20, small solid structures are present and can be detected by filtration at 0.45 μm. This suggests that precipitation may occur at slightly lower ion concentration than was detected by the turbidimetric procedure employed.

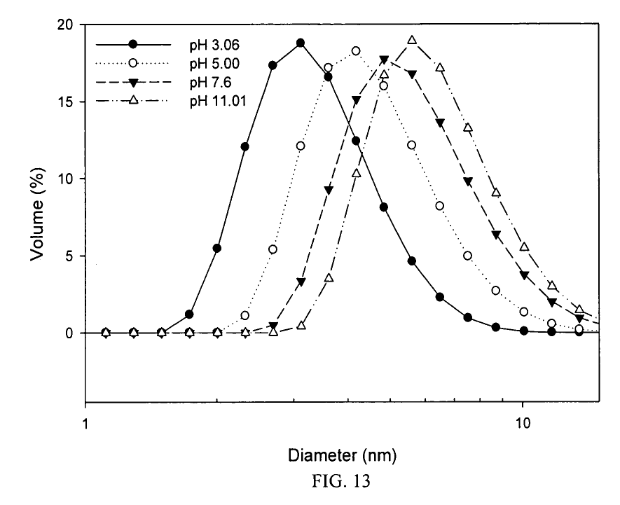

Without restriction to any one theory or mode of operation, such data supports the formation of mixed micelles comprising cationic and nonionic headgroups. As noticed for LAE pure micelles, mixed micelles grow with increased solution (FIG. 13). However, the effect is much less impressive. This may be because the micelles surface is not as strongly positively charged as in the pure micelles, since the nonionic molecules intercalate between the positive ones. The same expansion is observed with different volume fractions of T20 in the bulk medium.

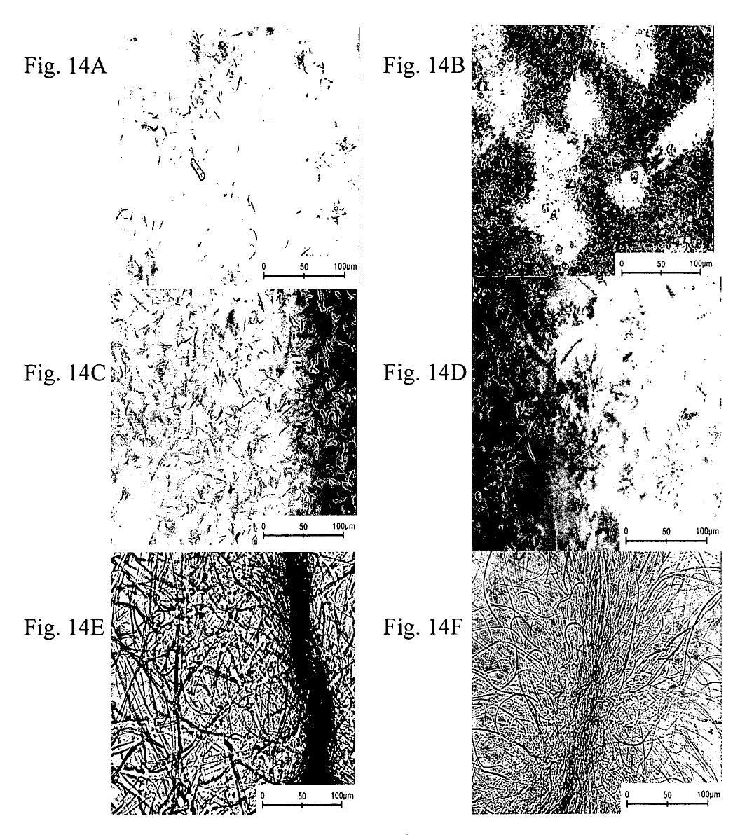

As shown on FIG. 14, precipitates appear different depending on their formation, the quantity of ions that has been added and the presence of nonionic surfactant. Indeed, when pH is increased above the threshold values, solid particles appear. Their appearance in the early states of precipitation consists of transparent, platelike crystals with a rectangular shape (A). Some of the solids displayed interference colors under polarized light. When higher pH values are reached, precipitates are made of little solid particles embedded in an amorphous and dispersed matrix, certainly containing tinier corpuscules (B). When T20 is added to the solutions before precipitation, new forms appear: thin filaments in the early states of precipitation (C). Later as pH increases, crystals appear (D).

After addition of salts, the micrographs reveal the presence of a network with “cores” surrounded by filaments (E and F). The same configuration is found with any concentration of salts provoking precipitation. Moreover, the pattern doesn't exhibit any modification with addition of T20.

As illustrated, above a representative ionic anti-microbial such as lauric arginate reacts in solution with added counterions or hydroxide ions to form solid structures. In contrast, the lower the water salinity and the lower the pH, the more stable the colloidal suspension. Increasing counterions concentration and pH results in more precipitation. The stability behavior of such surfactant micelles against these physico-chemical stresses can be enhanced by addition of a nonionic surfactant to the mixtures. A nonionic surfactant is likely to increase salinity tolerance in the way that can be thought of reducing repulsion between the ionic headgroups: promoting mixed micelles formation, decreasing the ionic monomer concentration, and reducing precipitation, this phenomenon.

More specifically, as relates to certain embodiments, LAE a cationic surfactant with broad spectrum antimicrobial functionality may precipitate from solution and loose activity under non-acidic conditions and in the presence of salts. Compositions comprising a nonionic surfactant in combination with lauric arginate can help overcome instability problems. Aqueous solutions containing 5 wt % LAE at pH 2-13 and at NaCl and CaCl₂ concentrations of up to 200 g/L were prepared with or without a nonionic emulsifier (e.g., Tween 20, 0.5-5 wt %). Turbidity was assessed spectrophotometrically. Formation of precipitates was quantified by filtration and their structure determined by optical microscopy. The critical micellar concentration (CMC) of stable dispersions was measured by drop tensiometry. Size and charge of micelles was measured using a laser light scattering technique. LAE precipitated from solution at pH>4.1 and at NaCl and CaCl₂ concentrations of >10 g/L and >15 g/L, respectively due to salt-induced electrostatic shielding and pH induced charge reduction. Precipitates had needle-like morphologies. Addition of Tween 20 increased stability of LAE in the presence of NaCl and CaCl₂ and at pH>4.1 depending on the concentration of the surfactant. For example, after addition of 0.5 wt % Tween 20, precipitates formed at >50 g/L NaCl and >200 g/L CaCl₂ and pH>11. Formation of precipitates was completely suppressed after addition of 5 wt % Tween 20. CMC and ζ-potential measurements indicated formation of a mixed micellar system. The CMC was of the mixed system was smaller compared to the single surfactant systems and the ζ-potential of micelles generally decreased upon addition of Tween 20. Such results suggest that addition of a nonionic surfactant to lauric arginate can improve stability to electrostatically-induced destabilizations thereby extending the range of applications in food systems. Likewise, such surface active and antimicrobial components and, the results shown, are representative of various broader aspects of this invention, benefits from which are available through use of a range of other surface active and antimicrobial components, as would be understood by those skilled in the art and aware of this invention.

### EXAMPLES OF THE INVENTION

The following non-limiting examples and data illustrate various aspects and features relating to the compositions, systems and/or methods of the present invention, including, preparation of compositions comprising various antibiotic/antimicrobial components and surfactant components, as can be achieved through the methodologies described herein. In comparison with the prior art, the present compositions, systems and/or methods provide results and data which are surprising, unexpected and contrary to the prior art. While the utility of this invention can be demonstrated through the use of several such compositions, it will be understood by those skilled in the art that comparable results are obtainable with various other antibiotic, antimicrobial, surfactant and emulsifier components, as are commensurate with the scope of this invention.

### Example 1a

As an example of the behavior encountered in the art, consider the lauric arginates. Addition of simple salts such as NaCl can lead to a reduction of the positive charge of the compound, which results in aggregation and phase separation and loss of antimicrobial activity. The lauric arginate solution that was initially transparent becomes turbid upon addition of the salt. Finally, as the large flocs sediment (move to the bottom), a highly turbid sediment phase and a clear supernatant phase can be observed.

### Example 1b

Demonstrating use of this invention, a clear solution of lauric arginate in deionized water can be prepared. Upon addition of HCl and NaOH, the dispersion becomes turbid indicating formation of large aggregates. Eventually these aggregates phase separate (not shown). Addition of a sufficient concentration of a nonionic surfactant, in this case polyoxyethylene (20) sorbitan monolaureate, restored stability of the dispersion leading to a transparent appearance.

### Example 2

As discussed above, various organic acids and their derivatives can provide antimicrobial activity. Representative of such components, consider benzoic acid. An increase in pH above the pKₐ can lead to proton dissociation, giving the compound a negative charge and resulting in loss of antimicrobial activity. The critical pH is typically around 4. As a further example of this invention, a composition comprising benzoic acid and polyoxyethylene (20) sorbitan monolaureate provides mixed micelle formation with overall reduced net charge. Such a system demonstrates, as compared to benzoic acid alone, improved interaction with microbial surfaces. As a result, higher antimicrobial activities are obtained, even at elevated pH.

With references to examples 3-6:

Materials and Solution Preparation. Concentrated Mirenat®-N solution (25% lauric arginate, 75% PEG) was obtained from A&B Ingredients and used without further purification. Polyoxyethylene 20 sorbitan monolaureate (Tween 20), solvents (HCl, NaOH, purity >99%) and NaCl (>99%) was obtained from Sigma Chemical (St. Louis, Mo.). All solutions were prepared using doubles distilled and deionized water. Micellar solutions were prepared by gently mixing surfactants (Tween and LAE) at appropriate concentrations with double distilled water. As needed, pH was adjusted using HCl and NaOH.

Measurement of pH. pH of all solutions was recorded using an Accumet model AR15 pH-meter (Fisher). The instrument was calibrated prior to measurements using appropriate buffer solution.

Solution Turbidity. Absorbance of solutions adjusted to pH or containing salts was measured by spectroscopy at a wavelength of 600 nm using a UV-Visible spectrophotometer (Spectronic 21D, Milton Roy, Rochester, N.Y.). All measurements were conducted at ambient temperature and repeated three times. The change in the of absorbance of solutions was used as an indirect measure to formation of larger particles an/or aggregates.

Precipitate Retention. 50 ml of micellar solutions containing precipitates was filtered through a Whatman 0.45 μm nylon filter. Filters were dried at 55° C. for 5 days and weight of dried precipitates measured using a balance. Precipitate Retention in percent was calculated as:

$$PR = \frac{{m}_{p}}{{m}_{s}} \qquad ( 1 )$$

where mₚ is the mass of precipitates and mₛ the mass of surfactant dispersed in the initial 50 ml of solution.

### Example 3

Microstructure of Precipitates. The microstructures of precipitates was assessed by optical microscopy 24 h after the pH of micellar solutions had been adjusted or salt had been added. A drop of solution containing precipitates was placed on a microscope slide and covered with a cover slip and then the microstructure was determined using optical microscopy (Nikon microscope Eclipse E400 with polarizer, Nikon Corporation, Japan). Images were acquired using a CCD camera (CCD-300-RC, DAGE-MTI, Michigan City, Ind.) connected to digital image processing software (Micro Video Instruments, Inc., Avon, Mass.) installed on a computer.

### Example 4

Measurement of Surface Tension. A drop shape analysis tensiometer (Model DSA-G10 MK2, Kruss USA, Charlotte, N.C.) was used to determine surface tension of surfactant solutions (20.0±0.5° C.). The tensiometer determines the shape of pendant drops or bubbles through numerical analysis of the entire drop shape. The calculation of the interfacial tension from the drop shape is based on the Young-Laplace equation of capillarity and a detailed description can be found elsewhere (Dukhin, Kretschmer et al. 1995). Surface tension measurements were carried out at 20.0±0.5 ° C. To ensure accurate temperature control, an air bubble was formed at the inverted tip of a syringe that was submerged in the surfactant solution contained in a thermostatted cuvette. The syringe/cuvette system was positioned on an optical bench between the light source and a high-speed CCD camera. The CCD camera was connected to a video frame-grabber board to record the image onto the hard-drive of a computer at a speed of 1 frame per 10 second to obtain the drop profile trough contour analysis. Samples were assumed to be equilibrated (the equilibrium dynamic interfacial tension DIT_{eq}) when measured values of DIT_{eq} remained unchanged for 30 minutes. The accuracy of surface tension measurements was ±0.2×10⁻³ N/m. Densities of solutions are required for the accurate determination of surface tension using the Young-Laplace equation. Solution were equilibrated to 20° C. in a waterbath and density measured using a digital density meter (DMA 35N, Anton Paar GmbH, Graz, Austria). The accuracy of density measurements using the DMA 35N was ±0.001 g/cm³. All measurements (density and surface tension) were repeated five times

### Example 5

Particle Size Analysis. A dynamic light scattering technique (NanoZS model ZEN3600, Malvern Instruments, MA) was used to determine the hydrodynamic radius of micelles. The instrument determines the size of micelles from the diffraction pattern with a 633 nm red laser and the detector set at 173°. The temperature was equilibrated to 25° C. The particle size measurements are reported as the mean diameter: d₄₃=Σnᵢdᵢ⁴/Σnᵢdᵢ³ or d₃₂=Σnᵢdᵢ³/Σnᵢdᵢ² where nᵢ is the number of droplets of diameter dᵢ. Each individual particle size measurement was determined from the average of three readings made on the same sample.

### Example 6

ζ-Potential. The ζ-potential of micellar solutions was measured using an electrophoretic technique. Samples were placed in a disposable cuvette that acted as the measurement chamber of the particle electrophoresis instrument (Zetasizer Nanoseries ZS, Malvern Instruments, Worcestershire, UK), and the ζ-potential was determined by measuring the direction and velocity that the droplets moved in the applied electric field. The Smoluchowsky mathematical model was used by the software to convert the electrophoretic mobility measurements into ζ-potential values. Each individual ζ-potential measurement was determined from the average of five readings made on the same sample.

While the principles of this invention have been described in connection with specific embodiments, it should be understood clearly that these descriptions are added only by way of example and are not intended to limit, in any way, the scope of this invention. For instance, in various broader respects, the present invention can comprise a variety of pharmaceutical, chemical, healthcare, food, cosmetic and personal care compositions, each of which comprising one or more of antimicrobial compositions of the sort described herein.

We claim:

1. A method of using a surface active component to maintain activity of an antimicrobial component, said method comprising:
providing an antimicrobial component in a medium; and
contacting said antimicrobial component with a surface active component, said surface active component in an amount at least partially sufficient to maintain the activity of said antimicrobial component over change in at least one of medium pH, temperature and ionic strength.

2. The method of claim 1 wherein said surface active component is selected from ionic and non-ionic surface active components and combinations thereof.

3. The method of claim 1 wherein said antimicrobial component is amphiphilic and at a concentration sufficient for micelle formation in said medium.

4. The method of claim 3 wherein said surface active component is amphiphilic and at a concentration at least one of sufficient for micelle formation and less than sufficient for micelle formation, in said medium.

5. The method of claim 4 wherein said antimicrobial component comprises an anionic portion, and said surface active component comprises a portion selected from cationic and non-ionic portions.

6. The method of claim 4 wherein said antimicrobial component comprises a cationic portion, and said surface active component comprises a portion selected from anionic and non-ionic portions.

7. The method of claim 6 wherein said antimicrobial component is a cationic amphiphile, and said surface active component is non-ionic.

8. A method of using a surface active component to maintain activity of an antimicrobial component, said method comprising:
providing an ionic amphiphilic antimicrobial component in an medium, said antimicrobial component at a concentration sufficient for micelle formation in said medium; and
contacting said antimicrobial component with an amphiphilic surface active component, said surface active component selected from ionic and non-ionic components and combinations thereof, at least one said surface active component in an amount at least partially sufficient to maintain the activity of said antimicrobial component.

9. The method of claim 8 wherein said surface active component is present in a concentration at least one of sufficient for micelle formation and less than sufficient for micelle formation, in said medium.

10. The method of claim 9 wherein said antimicrobial component comprises an anionic portion, and said surface active component comprises a portion selected from cationic and non-ionic portion.

11. The method of claim 9 wherein said antimicrobial component comprises an cationic portion, and said surface active component comprises a portion selected from anionic and non-ionic portions.

12. The method of claim 11 wherein said antimicrobial component is a cationic amphiphile, and said surface active component is non-ionic.

13. The method of claim 12 wherein said antimicrobial component is a lauric arginate salt.

14. The method of claim 12 wherein said surface active component is a polyoxyethylene sorbitan monolaureate.

15. An antimicrobial composition comprising an ionic amphiphilic antimicrobial component, an amphiphilic surface active component and a composition medium, at least one said component at a concentration sufficient for micelle formation in said medium.

16. The composition of claim 15 comprising an anionic amphiphilic antimicrobial component, and a surface active component selected from cationic surface active components, non-ionic surface active components and combinations thereof.

17. The composition of claim 15 comprising a cationic amphiphilic antimicrobial component, and a surface active component selected from anionic surface active components, non-ionic surface active components and combinations thereof.

18. The composition of claim 15 comprising a pharmaceutically acceptable carrier component.

19. The composition of claim 15 comprising a food grade medium, said composition applied to a food component.

20. An antimicrobial composition comprising a lauric arginate salt, an amphiphilic non-ionic surface active component and a composition medium, said arginate salt at a concentration sufficient for micelle formation in said medium.

21. The composition of claim 20 wherein said surface active component comprises a polyoxyethylene sorbitan monolaureate.

22. The composition of claim 21 on a food component.
